(19) United States
(12) Patent Application Publication
(10) Pub. No.: US 2019/0298953 A1
(43) Pub. Date: Oct. 3, 2019

(54) RAIN-OUT RESISTANT COMPONENT A GAS DELIVERY SYSTEM

(71) Applicant: KONINKLIJKE PHILIPS N.V., EINDHOVEN (NL)

(72) Inventors: Robert William BAIKO, Pittsburgh, PA (US); Mark AMARATUNGA, Pittsburgh, PA (US)

(73) Assignee: KONINKLIJKE PHILIPS N.V., EINDHOVEN (NL)

(21) Appl. No.: 16/307,245

(22) PCT Filed: Jun. 27, 2017
(86) PCT No.: PCT/EP2017/065899

§ 371 Date: Dec. 5, 2018

Related U.S. Application Data
(60) Provisional application No. 62/355,386, filed on Jun. 28, 2016.

(51) Int. Cl. A61M 16/06; A61M 16/00; A61M 16/10

(52) U.S. Cl. CPC A61M 16/0605; A61M 16/0003; A61M 2210/0625; A61M 2210/0618; A61M 16/10

(57) ABSTRACT

A component—such as a respitratory mask—for use in a user circuit for delivering a flow of a treatment gas to the airway of a patient includes a housing. The housing includes an exterior portion having a number of exterior surfaces positioned to be exposed to a surrounding environment. The housing further includes an interior portion having a number of interior surfaces (30) defining a passageway through the housing. The exterior portion has a first heat capacity and the interior portion has a second heat capacity which is less than the first heat capacity.

### CROSS-REFERENCE TO RELATED APPLICATIONS

This patent application claims the priority benefit under 35 U.S.C. § 119(e) of U.S. Provisional Application No. 62/355,386 filed on Jun. 28, 2016, the contents of which are herein incorporated by reference.

### BACKGROUND OF THE INVENTION

### 1. Field of the Invention

The present invention pertains to systems for delivering a flow of breathing gas to the airway of a patient and more particularly to components for use in a user circuit for such delivery. The present invention also pertains to user circuits for delivering a flow of breathing gas to the airway of a patient.

### 2. Description of the Related Art

There are numerous situations where it is necessary or desirable to deliver a flow of breathing gas non-invasively to the airway of a patient, i.e., without intubating the patient or surgically inserting a tracheal tube in their esophagus. For example, it is known to ventilate a patient using a technique known as non-invasive ventilation. It is also known to deliver positive airway pressure (PAP) therapy to treat certain medical disorders, the most notable of which is OSA. Known PAP therapies include continuous positive airway pressure (CPAP), wherein a constant positive pressure is provided to the airway of the patient in order to splint open the patient's airway, and variable airway pressure, wherein the pressure provided to the airway of the patient is varied with the patient's respiratory cycle. Such therapies are typically provided to the patient at night while the patient is sleeping.

Non-invasive ventilation and pressure support therapies as just described involve the placement of a patient interface device including a mask component having a soft, flexible cushion on the face of a patient. The mask component may be, without limitation, a nasal mask that covers the patient's nose, a nasal cushion having nasal prongs that are received within the patient's nares, a nasal/oral mask that covers the patient's nose and mouth, or a full face mask that covers the patient's face. Such patient interface devices may also employ other patient contacting components, such as forehead supports, cheek pads and chin pads. The patient interface device is connected to a gas delivery tube or conduit and interfaces the ventilator or pressure support device with the airway of the patient, so that a flow of breathing gas can be delivered from the pressure/flow generating device to the airway of the patient.

During such therapies, it is common for the delivered breathing gas to be supplemented with humidified air. With humidified air there comes a risk for anything touching the humidified air to thermally cool the air and cause the water therein to rain out or condensate on the surface which is contacted. The reason for such condensation, or “rain out” as such occurrence is commonly coined in the market, is the drop in temperature of the humidified air. When the moist high humidity air touches a surface that is significantly cooler than the air at its current state, the humidified air will go from for example approximately 85% relative humidity at 32C to 27C. Such drop in temperature will cause the relative humidity to increase past 100%, meaning that any remaining moisture in the air will fall from suspension (rain) or condensate on any surfaces colder than it. FIG. 1 is a chart showing the relationship between humidity and temperature.

In addition to condensation or “rain out” resulting from a humidified supply of breathing gas, such occurrence may also result from warm humid air expelled by a patient into the system. The droplets of water resulting from either source can pool up and pose a risk for the patient as well as an uncomfortable feeling depending on the location of the pooling. Such conditions are exacerbated as either the humidity and/or the temperature of the treatment gas is increased, which is especially problematic as increasing either or both the humidity and/or temperature of the treatment gas is generally desirable to the patient.

A solution to address such rain out is to actively heat components in the gas delivery system through the use of heating elements. Such solution, however, is typically complex, expensive, and of questionable reliability.

### SUMMARY OF THE INVENTION

Accordingly, embodiments of the present invention provide user circuits and/or components thereof which reduce the occurrence of rain out in a system for delivering a flow of breathing gas to the airway of a patient. As one aspect of the present invention, a component for use in a user circuit for delivering a flow of a treatment gas to the airway of a patient comprises a housing. The housing comprises: an exterior portion having a number of exterior surfaces positioned to be exposed to a surrounding environment; and an interior portion having a number of interior surfaces defining a passageway through the housing. The exterior portion has a first heat capacity, and the interior portion has a second heat capacity which is less than the first heat capacity.

The interior portion may include a plurality of structures disposed in close proximity on one of the number of interior surfaces and each structure may extend from the respective interior surface into the passageway. The plurality of structures may comprise a plurality of fins. The plurality of structures may comprise a plurality of finger-like projections. The plurality of structures may comprise a plurality of elements formed as a surface texture. The plurality of structures may comprise at least two of the group consisting of: a fin, a finger-like projection, and a surface texture.

The housing may comprise a polished outer surface. The housing may comprise an insulating coating disposed on at least one exterior surface of the number of exterior surfaces. The housing may comprise a conduit. The housing may comprise a fluid coupling conduit. The housing may comprise a portion of a patient interface device.

As another aspect of the present invention, a patient circuit for use in delivering a flow of treatment gas to an airway of a patient comprises: a delivery conduit having a first end structured to be coupled to a pressure generating device and an opposite second end; a patient interface device structured to sealing engage the patient; and a fluid coupling conduit coupled between the second end of the delivery conduit and the patient interface device. At least one of the delivery conduit, the patient interface device or the fluid coupling conduit include a housing comprising: an exterior portion having a number of exterior surfaces positioned to be exposed to a surrounding environment; and an interior portion having a number of interior surfaces defining a passageway through the housing. The exterior portion has a first heat capacity, and the interior portion has a second heat capacity which is less than the first heat capacity.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a chart showing the relationship between humidity and temperature.

FIG. 2 is a partially schematic view of a system adapted to provide a regiment of respiratory therapy to a user according to an exemplary embodiment of the present invention;

FIG. 3 is an example of a fin arrangement according to an exemplary embodiment of the present invention;

FIG. 4 is an example of a finger-like projection arrangement according to an exemplary embodiment of the present invention;

FIG. 5 is an example of a surface arrangement according to an exemplary embodiment of the present invention; and

FIG. 6 is an example of another surface arrangement according to an exemplary embodiment of the present invention.

### DETAILED DESCRIPTION OF EXEMPLARY EMBODIMENTS

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components. As employed herein, the term “number” shall mean one or an integer greater than one (i.e., a plurality).

As used herein, the term “patient” or “user” shall be used interchangeably to refer to the person to which the interface device is delivering a flow of breathing gas. As used herein, the term “heat capacity” shall be used to refer to the measureable physical quantity of an arrangement which is equal to the ratio of the heat added to an arrangement to the resulting temperature change of the surface of the arrangement. For example, if two arrangements are exposed to an environment at a given temperature, the arrangement having a lower heat capacity will have a higher surface temperature than the arrangement having a higher heat capacity. Heat transfer to an arrangement may occur via one or more of: conduction, convention, and radiation.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

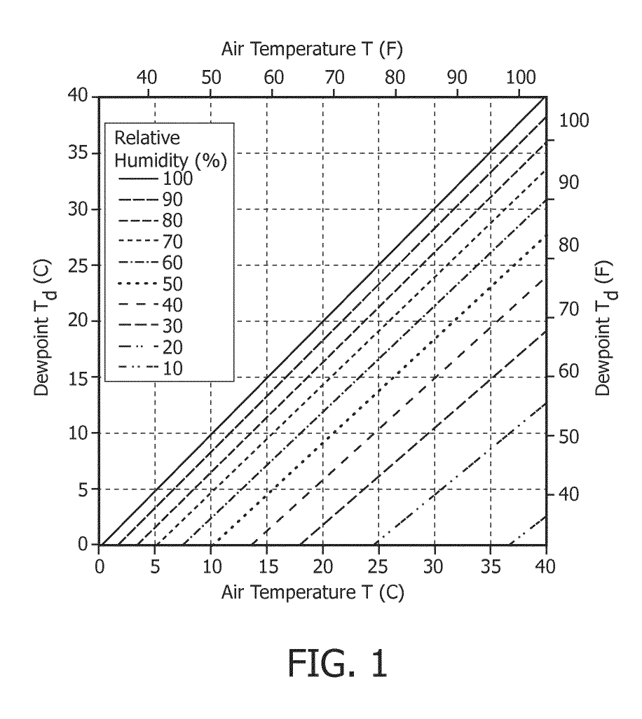

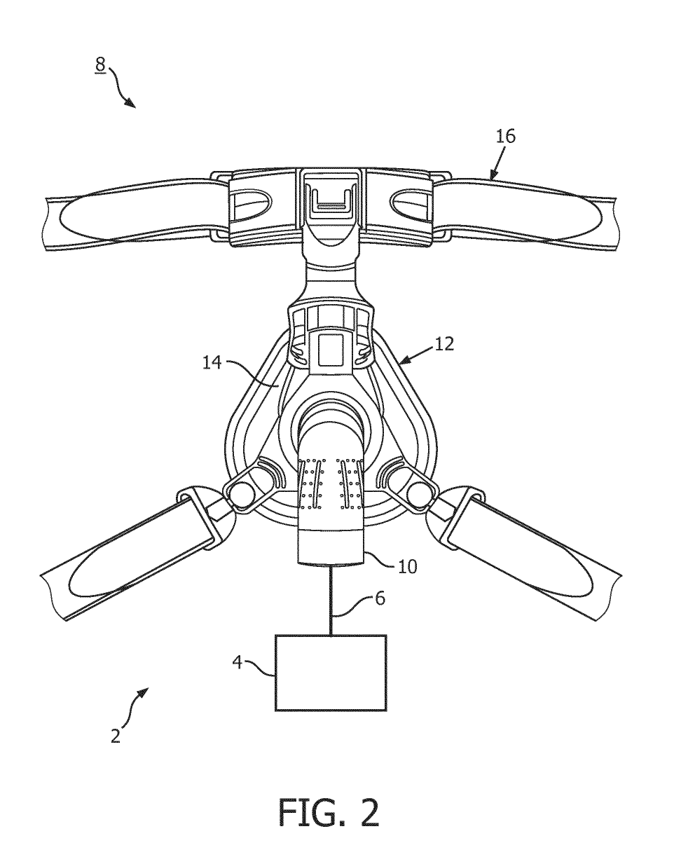

A system 2 adapted to provide a regimen of respiratory therapy to a patient according to one exemplary embodiment of the invention is generally shown in FIG. 1. System 2 includes a pressure generating device 4 (shown schematically), a delivery conduit 6 (shown partially schematically), and a patient interface device 8 having a fluid coupling conduit 10 coupled thereto. Pressure generating device 4 is structured to generate a flow of breathing gas and may include, without limitation, ventilators, constant pressure support devices (such as a continuous positive airway pressure device, or CPAP device), variable pressure devices (e.g., BiPAP®, Bi-Flex®, or C-Flex™ devices manufactured and distributed by Philips Respironics of Murrysville, Pa.), and auto-titration pressure support devices. Delivery conduit 6 is structured to communicate the flow of breathing gas from pressure generating device 4 to user interface device 8 through a fluid coupling conduit 10, which in the illustrated embodiment is an elbow connector. Delivery conduit 6, fluid coupling conduit 10 and user interface device 8 are often collectively referred to as a user circuit.

A BiPAP® device is a bi-level device in which the pressure provided to the patient varies with the patient's respiratory cycle, so that a higher pressure is delivered during inspiration than during expiration. An auto-titration pressure support system is a system in which the pressure varies with the condition of the patient, such as whether the patient is snoring or experiencing an apnea or hypopnea. For present purposes, pressure/flow generating device 4 is also referred to as a gas flow generating device, because flow results when a pressure gradient is generated. The present invention contemplates that pressure/flow generating device 4 is any conventional system for delivering a flow of gas to an airway of a user of interface device 8 or for elevating a pressure of gas at an airway of the user, including the pressure support systems summarized above and non-invasive ventilation systems.

In the exemplary embodiment illustrated in FIG. 1, user interface device 8 is depicted as a nasal mask which includes a user sealing assembly or cushion 12 coupled to a generally rigid frame 14 which is coupled to conduit 6 via fluid coupling conduit 10. However, it is to be appreciated that other types of patient interface devices, such as, without limitation, a nasal pillows mask, an oral mask, a nasal/oral mask, or a full/total face mask (which encompasses the entire face), which facilitates the delivery of the flow of breathing gas to the airway of a user, may be substituted for user interface device 8 while remaining within the scope of the present invention. It is also to be appreciated that conduit 6 may be directly coupled to user interface device 8 without the use of any intermediary coupling, such as conduit 10. User interface device 8 may be secured to the head of a user via a suitable headgear 16.

In order to reduce the occurrence of undesired condensation or rain out (such as previously discussed) in the user circuit, embodiments of the present invention utilize heat from the flow of breathing gas to heat interior portions, and more particularly interior surfaces, of the user circuit. In general, such object is carried out by promoting heat transfer from the flow of breathing gas passing along the passageway formed within the components (e.g., delivery conduit 6, fluid coupling conduit 10, user interface device 8) of the user circuit into the interior surfaces of such components. By promoting such heat transfer, temperature differences between the flow and interior surfaces are minimized, thus eliminating rain out. Such promoting of heat transfer is accomplished in embodiments of the present invention by reducing the heat capacity of the interior portions/surfaces which are exposed to the flow as compared to exterior portions/surfaces which are exposed to the environment. Conversely, the heat capacity of the exterior portions/surfaces may be increased as compared to the interior portions/surfaces.

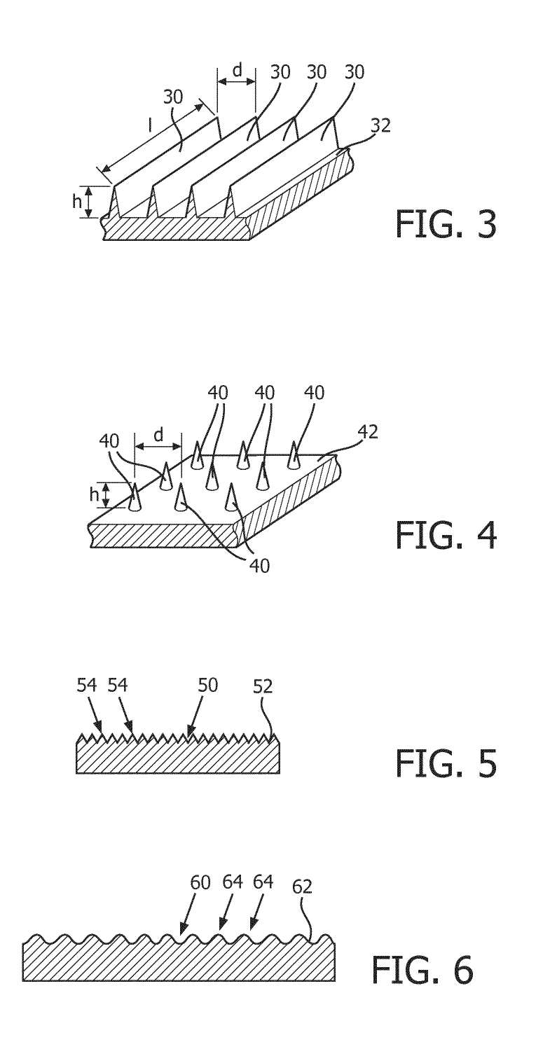

FIGS. 3-6 illustrate some example arrangements having a reduced heat capacity which may be employed to promote heat transfer from the flow of warm humid air passing through a component (be it the flow of breathing gas from a pressure generating device or exhalation gas from a user) into the particular component in accordance with example embodiments of the present invention. As shown in FIG. 3, a plurality of fins 30 may be disposed so as to extend a height h from an interior surface 32 of a housing which generally forms one or more of the components of the user circuit. In example embodiments of the present concept, fins 30 having a height in the range of 1-5 mm have been employed, however other heights may be employed without varying from the scope of the present invention. Each fin 30 may extend a length l and be spaced (center-to-center) from an adjacent fin 30 by a separation distance d. One or more of fins 30 may be formed separately from, and subsequently coupled to interior surface 32. Alternatively, fins 30 may be integrally formed with interior surface 32.

Some examples of suitable materials from which fins 30 may be formed include, for example, without limitation, polycarbonates, thermal plastic elastomers, thermoplastics and silicone parts (e.g., silicone, thermoplastic elastomer, vinyl, elastomeric material). Generally materials that have a higher thermal conductivity (property associated with the ability to transfer temperature across unit thickness) are desired compared to materials that have a low thermal conductivity (insulators) that would be preferred for use on the outside portions. Although shown as extending in a generally straight manner, it is to be appreciated that such fins 30 may be of different shapes without varying from the scope of the present invention. For example, without limitation, curved sections that are not straight but follow the curve of the mask may be employed. The sections do not have to be perpendicular or in line with flow. Generally when faceplates are made they have certain draft and direction of features so the angle of the fin protruding from the surface could be in any orientation. In example embodiments of the present invention, fins which are disposed parallel or generally parallel provide adequate heat transfer without adversely affecting the flow of treatment gas. It is also to be appreciated that the cross-sectional shape, which is shown as tapering in the example of FIG. 3, may be varied without varying from the scope of the present invention. From the foregoing, it is thus to be appreciated that generally any arrangement which does not adversely affect noise, pressure drop, or flow may be employed without varying from the scope of the present invention.

As shown in FIG. 4, a plurality of finger-like projections 40 may be disposed so as to extend a height h from an interior surface 42 of a housing which generally forms one or more of the components of the user circuit. Each projection 40 may be spaced (center-to-center) from an adjacent projection 40 by a separation distance d. One or more of projections 40 may be formed separately from, and subsequently coupled to interior surface 42. Alternatively, projections 40 may be integrally formed with interior surface 42. Although shown as being generally conical, it is to be appreciated that projections 40 may be of different shapes (e.g., without limitation, cylinders, pyramids, points, squares, hexagonal, polygonal, abstract, etc.) without varying from the scope of the present invention.

As shown in FIGS. 5 and 6, an interior surface 52, 62 of one or more of the components of the user circuit may be provided with a surface texture 50, 60 which promotes heat transfer into the surface 52, 62. As shown in the example of FIG. 5, such texture 50 may have a number of sharp edges 54 (only two of which are labeled) which project outward from surface 52. As shown in the example of FIG. 6, such texture 60 may have a number of smooth mounds 64 (only two of which are labeled) which project outward from surface 62. In another example, a texture consisting of a plurality of random, non-sinusoidal shapes, formed by sandblasting has been employed. It is to be appreciated that the textures described herein are provided for example purposes only and that other textures may be employed without varying from the scope of the present invention.

In addition to modifying the interior surface of one or more of the components of the user circuit, one or more of the exterior surfaces of such components may also be modified to reduce heat transfer from the component to the surrounding environment (which would cool the component). Such modifications may include, without limitation, polishing the exterior surface (thus reducing the surface areas thereof) and/or coating the exterior surface with an insulating coating.

Although shown as being generally planar, it is to be appreciated that the interior surfaces 32, 42, 52, 62 described herein are illustrated for exemplary purposes only and that the interior surfaces to which the concepts described herein may be of various shapes (e.g., without limitation, curved) without varying from the scope of the present invention.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment. Additionally, although the invention has been described in conjunction with a user interface device for delivering a flow of treatment gas to a user, it is to be appreciated that embodiments of the present invention may be utilized in other applications wherein strap members are employed without varying from the scope of the present invention.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

1. A component for use in a user circuit for delivering a flow of a treatment gas to the airway of a patient, the component comprising:
a housing comprising:
an exterior portion having a number of exterior surfaces positioned to be exposed to a surrounding environment; and
an interior portion having a number of interior surfaces defining a passageway through the housing, wherein the interior portion includes a plurality of projections suitable for heat transfer disposed in close proximity on one of the number of interior surfaces, wherein each projection extends from the respective interior surface into the passageway, and wherein the exterior portion has a first heat capacity, and wherein the interior portion has a second heat capacity which is less than the first heat capacity.

2. (canceled)

3. The component of claim 1, wherein the plurality of structures comprises a plurality of fins.

4. The component of claim 1, wherein the plurality of structures comprises a plurality of finger-like projections.

5. The component of claim 1, wherein the plurality of structures comprises a plurality of elements formed as a surface texture.

6. The component of claim 1, wherein the plurality of structures comprises at least two of the group consisting of: a fin, a finger-like projection, and a surface texture.

7. The component of claim 1, wherein the housing comprises a polished outer surface.

8. The component of claim 1, wherein the housing comprises an insulating coating disposed on at least one exterior surface of the number of exterior surfaces.

9. The component of claim 1, wherein the housing comprises a conduit.

10. The component of claim 1, wherein the housing comprises a fluid coupling conduit.

11. The component of claim 1, wherein the housing comprises a portion of a patient interface device.

12. A patient circuit for use in delivering a flow of treatment gas to an airway of a patient, the patient circuit comprising:
a delivery conduit having a first end structured to be coupled to a pressure generating device and an opposite second end;
a patient interface device structured to sealing engage the patient; and
a fluid coupling conduit coupled between the second end of the delivery conduit and the patient interface device,
wherein at least one of the delivery conduit, the patient interface device or the fluid coupling conduit include a housing comprising:
an exterior portion having a number of exterior surfaces positioned to be exposed to a surrounding environment; and
an interior portion having a number of interior surfaces defining a passageway through the housing, wherein the exterior portion has a first heat capacity, wherein the interior portion includes a plurality of projections suitable for enhancing heat transfer disposed in close proximity on one of the number of interior surfaces, wherein each structure extends from the respective interior surface into the passageway, and wherein the interior portion has a second heat capacity which is less than the first heat capacity.

13. (canceled)

14. The patient circuit of claim 12, wherein the plurality of structures comprises a plurality of fins.

15. The patient circuit of claim 12, wherein the plurality of structures comprises a plurality of finger-like projections.

16. The patient circuit of claim 12, wherein the plurality of structures comprises a plurality of elements formed as a surface texture.
